(19) United States
(12) Patent Application Publication
(10) Pub. No.: US 2011/0213261 A1
(43) Pub. Date: Sep. 1, 2011

(54) SYSTEMS AND METHODS FOR USE WITH SUBCUTANEOUS IMPLANTABLE MEDICAL DEVICES FOR DETECTING ELECTRODE/TISSUE CONTACT PROBLEMS

(76) Inventors: Mihir Naware, San Jose, CA (US); Cecilia Qin Xi, San Jose, CA (US)

(21) Appl. No.: 12/714,231

(22) Filed: Feb. 26, 2010

(51) Int. Cl. A61B 5/0452

(52) U.S. Cl. 600/515

(57) ABSTRACT

Techniques are provided for detecting problems involving electrode/tissue contact with extracardiac electrodes of subcutaneous monitoring devices, such as atrial fibrillation (AF) monitors. Briefly, subcutaneous impedance signals are detected using extracardiac sensing electrodes of the subcutaneous device. Problems involving poor electrode/tissue contact are then detected within the subcutaneous impedance signals. Depending upon its programming, the device can then inhibit the recording of subcutaneous electrocardiogram (ECG) data during periods of poor contact. Additionally, the device can identify the particular contact problem based on the impedance signals. In one example, the device identifies one or more of: acute instability of impedance indicative of intermittent electrode/tissue contact; impedance signal saturation indicative of loss of electrode/tissue contact; and impedance signal dropout indicative of the presence of liquids surrounding the electrodes (such as blood or edema accumulation.) Techniques for programming various modes of operation of the subcutaneous device are also provided.

### FIELD OF THE INVENTION

The invention generally relates to subcutaneous implantable medical devices—such implantable cardiac monitors (ICMs) or subcutaneous atrial fibrillation (AF) monitors—and to techniques for detecting and responding to problems caused by poor contact between extracardiac electrocardiac signal sensing electrodes and adjacent patient tissues.

### BACKGROUND OF THE INVENTION

An ICM is a diagnostic tool implanted beneath patient skin that monitors patient electrocardiogram (ECG) signals using extracardiac, subcutaneous (subQ) electrodes and records the ECGs for episodes of arrhythmias, such as asystole, bradycardia, tachycardia, and AF. Implantable AF monitors are devices particularly directed to detecting and diagnosing AF. Other subcutaneous devices adapted for detecting and recording ECGs include implantable loop recorders.

AF is a very common supraventricular tachycardia (SVT) that is a leading risk factor for ischemic stroke. However, AF is often asymptomatic and intermittent, which can result in inappropriate diagnosis and/or in delay in proper treatment. To address this problem, many ICMs now monitor for AF. For example, ECG signals detected by the extracardiac electrodes of the device are monitored by the device to detect R-waves (also referred to herein as QRS-complexes), which are representative of the depolarization of the ventricles. Due to high atrial rate of AF, not every atrial beat can be conducted to ventricle, often resulting in irregular intervals between R-waves. Therefore, the device assesses RR interval variability and detects an episode of AF based, e.g., on a significant increase of that variability.

Unfortunately, a common problem with subcutaneous ICMs and AF monitors is inferior ECG signal quality due to poor electrode/tissue contact, which can result in artifacts such as noise, signal dropout, signal saturation or baseline wander. In the presence of bad input signals, the device could falsely detect AF or other arrhythmias. Such false detections can swamp the limited episode storage capabilities of the device and overwrite legitimate episodes. From the perspective of the marketability of the device, this further leads to speculation by clinicians about the accuracy of detection, especially if the clinician is presented with a large number of falsely detected episodes and noisy signals. Still father, poor quality signals can prevent the device from properly detecting actual episodes of AF.

Accordingly, it would be highly desirable to provide techniques for detecting and qualifying periods of poor electrode/tissue contact and for responding accordingly.

### SUMMARY OF THE INVENTION

In an exemplary embodiment, a method is provided for use with an implantable subcutaneous medical device for subcutaneous implant within a patient wherein the device employs extracardiac sensing electrodes for contact with patient tissue. Briefly, the method includes detecting subcutaneous impedance signals using the extracardiac sensing electrodes of the subcutaneous device and then detecting an indication of poor electrode/tissue contact within the subcutaneous impedance signals. At least one device function is then controlled in response the indication of poor electrode/tissue contact, such as inhibiting the recording of subcutaneous ECG data by the device or storing diagnostic information qualifying the nature of the poor contact.

In an illustrative embodiment, the subcutaneous medical device is AF monitor equipped to detect patient R-waves for the purposes of detecting episodes of AF within the patient. Based on the timing of the R-waves, the device tracks ventricular refractory periods and, in one example, delivers impedance detection pulses of relatively large magnitude during the refractory periods (when the pulses will not interfere with the detection of events within the ECG.) Impedance is then measured by the device based on electrical signals detected in response to the impedance detection pulses. In another example, a stream of impedance detection pulses is delivered both within and outside of the refractory periods at sufficiently low amplitudes to avoid crosstalk with sensed ECG signals.

Insofar as detecting an indication of poor electrode/tissue contact is concerned, the device is preferably equipped to identify one or more of: acute instability of impedance; impedance signal saturation (unacceptably high impedance); and impedance signal dropout (unacceptably low impedance). Based on these particular issues, the device then qualifies the electrode/tissue contact problem. For example, acute instability of impedance is deemed to be indicative of intermittent electrode/tissue contact. Unacceptably high impedance is deemed to be indicative of loss of electrode/tissue contact. Unacceptably low impedance is deemed to be indicative of the presence of liquids surrounding the electrodes (such as blood or edema accumulating during an acute post-implant recovery phase.) Diagnostic indicators, such as “event markers,” are then stored in memory for transmission to an external device. The markers identify the particular electrode/tissue contact problem for clinician review. Herein, these diagnostic indicators are also referred to as warnings as they serve to warn the clinician of the poor electrode/tissue contact.

In response to the detection of poor electrode/tissue contact, the device can take various actions, depending upon its current programming. For example, the device can inhibit the storage of subcutaneous ECG data during an interval of poor electrode/tissue contact, so as to avoid consuming memory with poor quality ECG data. As another example, the device can inhibit the identification of arrhythmias such as AF during an interval of poor electrode/tissue contact, so as to avoid false detections. Still further, the device can record histograms of subcutaneous impedance data so as to track or trend the data over time to assess changes in impedance due to implant pocket maturation or other factors and to also reveal sudden acute changes in the pocket that could require reassessment by the clinician.

When used in conjunction with an external device, such as a programming device, a clinician can program the operation of the subcutaneous device to operate in various pre-specified modes of operation. For example, the clinician can program the device to operate in an “INHIBITORY” mode wherein the device inhibits the recording of ECGs and the detection of AF during periods of poor electrode/tissue contact. As another example, the clinician can program the device to operate in a “MONITORING” mode wherein the device monitors for periods of poor electrode/tissue contact by providing appropriate markers but does not otherwise inhibit operations in response to the periods. As yet another example, the clinician can program the device to operate in a “NORMAL” mode wherein the device does not monitor for periods of poor electrode/tissue contact and hence does not inhibit operations in response to any such periods. This latter mode might be used, e.g., when the clinician knows that electrode/tissue contact within the particular patient has already been shown to be sufficiently reliable.

Method and system examples are described in detail below.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 illustrates a subcutaneous ECG-based AF/ECG monitor system capable of detecting and qualifying poor electrode/tissue contact issues by using impedance signals;

FIG. 2 provides an overview of the method for detecting and qualifying poor electrode/tissue contact performed by the system of FIG. 1;

FIG. 3 illustrates an exemplary embodiment of the general technique of FIG. 2, wherein the subcutaneous device operates to selectively inhibit various functions—such as the recording of ECG data—in response to the detection of poor electrode/tissue contact;

FIG. 4 provides graphs illustrating noise, saturation and baseline wander in ECG signals;

FIG. 5 provides graphs of simulated results illustrating the impact of signal dropout on ECG signals, particularly illustrating a failure to detect R-waves that can result in a false detection of bradycardia;

FIG. 6 provides graphs of simulated results illustrating the impact of baseline wander and noise on ECGs, particularly illustrating circumstances where intermittent detection of R-waves might result in false detection of AF;

FIG. 7 illustrates an embodiment wherein a clinician using an external device programs the subcutaneous device of FIG. 1 to operate in one of several programmable modes of operation; and

FIG. 8 is a functional block diagram of the subcutaneous ECG/AF monitor of FIG. 1, illustrating basic device circuit elements that monitor for AF and that detect electrode/tissue contact problems based on impedance.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators are used to refer to like parts or elements throughout.

Overview of Implantable subQ Monitor

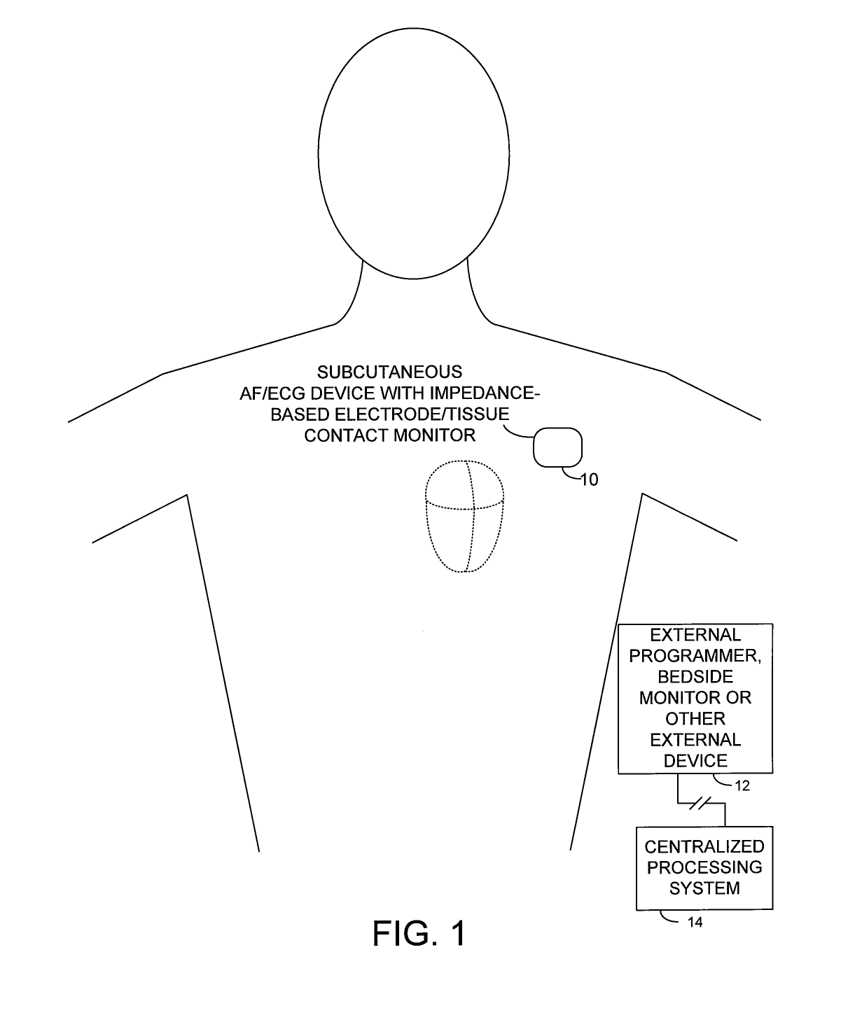

Referring first FIG. 1, a broad overview of components of an exemplary embodiment of the invention will now be provided. Briefly, FIG. 1 illustrates a subcutaneously-implanted leadless “subQ” AF/ECG monitor or ICM 10 equipped to detect and record ECG data, as well as to detect electrode/tissue contact problems that can result, e.g., in baseline wander, saturation or signal dropout within the ECG signal. The electrode/tissue contact problems are detected based on subcutaneous impedance signals. The subQ monitor also includes components for detecting episodes of AF or other arrhythmias and for recording ECGs and other data during the episodes. An external programmer, bedside monitor, or other external device 12 can be used to download the ECG data for display and to reprogram the operation of the subQ device.

As will be explained, the AF/ECG monitor can be programmed to selectively inhibit the recording of ECG data during periods of poor electrode/tissue contact and to store event markers to identify those periods. Additionally, the subQ device is equipped to qualify the contact problems (i.e. to identify the particular contact problem that is occurring) and to store suitable diagnostic indicators or warnings. A clinician operating the external programmer can then examine the diagnostic information and/or warnings to determine the source of the contact problem and then take appropriate remedial action, such as by adjusting the location of the subQ device within the patient.

Note that the external device can also be equipped to forward diagnostic information/warnings via a centralized processing system 14 to the patient's primary care physician for remote display. This is particularly useful in implementations where the external device is a bedside monitor. The centralized system may include such systems as Merlin.Net of St. Jude Medical, which may be used in conjunction with bedside monitors or similar devices such as the HouseCall™ remote monitoring system or the Merlin@home systems, also of St. Jude Medical.

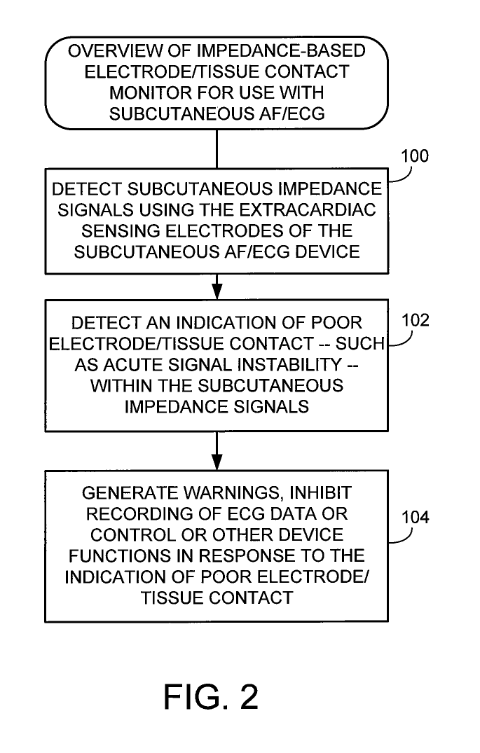

FIG. 2 broadly summarizes the electrode/tissue contact monitoring techniques performed by the subQ monitor of FIG. 1 or other suitably-equipped devices. At step 100, the subQ monitor detects subcutaneous impedance signals using its extracardiac sensing electrodes. At step 102, the subQ monitor detects an indication of poor electrode/tissue contact—such as acute signal instability—within the subcutaneous impedance signals. At step 104, the subQ monitor generates warnings, inhibits recording of ECG data or controls or other device functions in response to the indication of poor electrode/tissue contact.

### Exemplary Electrode/Tissue Contact Monitoring Techniques

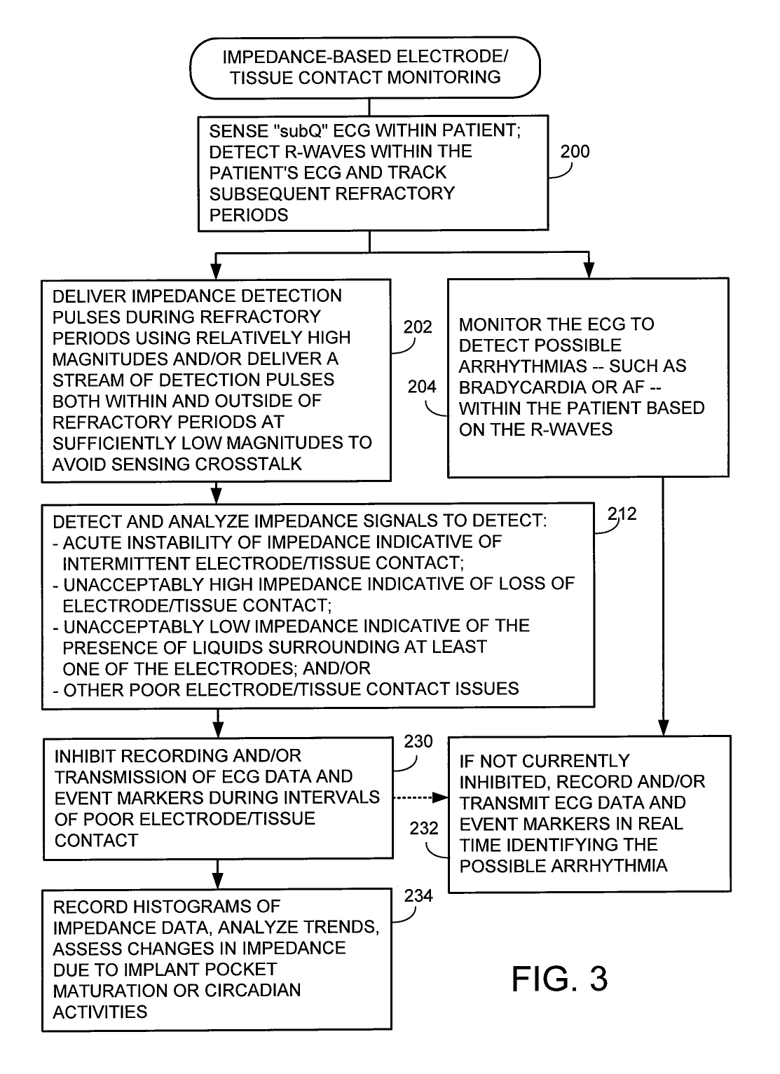

FIG. 3 illustrates an exemplary electrode/tissue contact monitoring technique that operates to qualify the nature of the electrode/tissue contact problem and to respond accordingly. At step 200, the subQ AF/ECG monitoring device senses a subcutaneous ECG signal within the patient and detects R-waves (i.e. QRS-complexes) within the patient's ECG. The device also tracks subsequent refractory periods, which are typically defined within the firmware of the device and extend, for example, for 200 ms following the R-wave. At step 202, the device (1) delivers impedance detection pulses during the refractory periods using relatively high magnitudes and/or (2) delivers a stream of detection pulses both within and outside of refractory periods at sufficiently low magnitudes to avoid sensing crosstalk with the ECG signals. The stream of impedance data thus collected can be used to perform an ongoing assessment of the electrode-tissue contact. Otherwise routine experimentation can be used to determine preferred or optimal magnitudes for the impedance detection pulses. In one particular example, a magnitude of 750 microAmps (μA) might be used during ventricular refractory periods. A magnitude of 250 μA might be used outside of the refractory periods without substantial risk of crosstalk. Note also that pulse width can affect the amount of crosstalk. In this regard, wider impedance detection pulses (e.g. 19 μsec) typically introduce more crosstalk than narrower pulses (e.g. 14 μsec.)

For leadless subQ devices, the impedance detection pulses can be delivered between an extracardiac “header” electrode mounted to (or integrated with) the housing of the device and a return “can” electrode also mounted to (or integrated with) the housing of the device. For devices with leads, the impedance detection pulse can be delivered, e.g., between a pair of subQ sensing electrodes mounted to one or more of extracardiac leads connected to the device. In any case, the electrodes are preferably positioned by a clinician during implant so as to provide proper electrode/tissue contact to allow for the reliable detection of ECG signals. As already explained, though, problems with electrode/tissue contact can nevertheless arise.

Note that a particularly effective tri-phasic impedance detection pulse for use in detecting impedance is described in U.S. patent application Ser. No. 11/558,194 of Panescu et al., filed Nov. 9, 2006, entitled “Closed-Loop Adaptive Adjustment of Pacing Therapy based on Cardiogenic Impedance Signals Detected by an Implantable Medical Device.” However, other impedance detection pulses or waveforms may instead be exploited.

Note also that, rather than detecting impedance, other related electrical signals or parameters can instead be exploited, such as admittance, conductance, immittance or their equivalents. This depends, in part, on how these parameters are defined. Impedance is the numerical reciprocal of admittance. Conductance is the numerical reciprocal of resistance. In general, impedance and admittance are vector quantities, which may be represented by complex numbers (having real and imaginary components.) The real component of impedance is resistance. The real component of admittance is conductance. When exploiting the real components of these values, conductance can be regarded as the reciprocal of impedance. Likewise, when exploiting the real components, admittance can be regarded as the reciprocal of resistance. Immittance represents either impedance or admittance. Generally, herein, “impedance signals” broadly encompasses impedance and/or any of these electrical equivalents and those skilled in the art can readily covert one such parameter to another.

Concurrently, at step 204, the subQ device monitors the ECG to detect possible arrhythmias—such as bradycardia or AF—within the patient based on the R-waves. Otherwise conventional arrhythmia detection techniques can be used, such as AF detection techniques based on RR interval variability or bradycardia detection techniques based on a ventricular rate derived from the R-waves. For AF detection, see, U.S. Patent Application 2009/0264783 of Xi et al., entitled “Systems and Methods for Improved Atrial Fibrillation (AF) Monitoring.” As noted, electrode/tissue contact problems can affect the capability of the device to reliably detect AF or other arrhythmias.

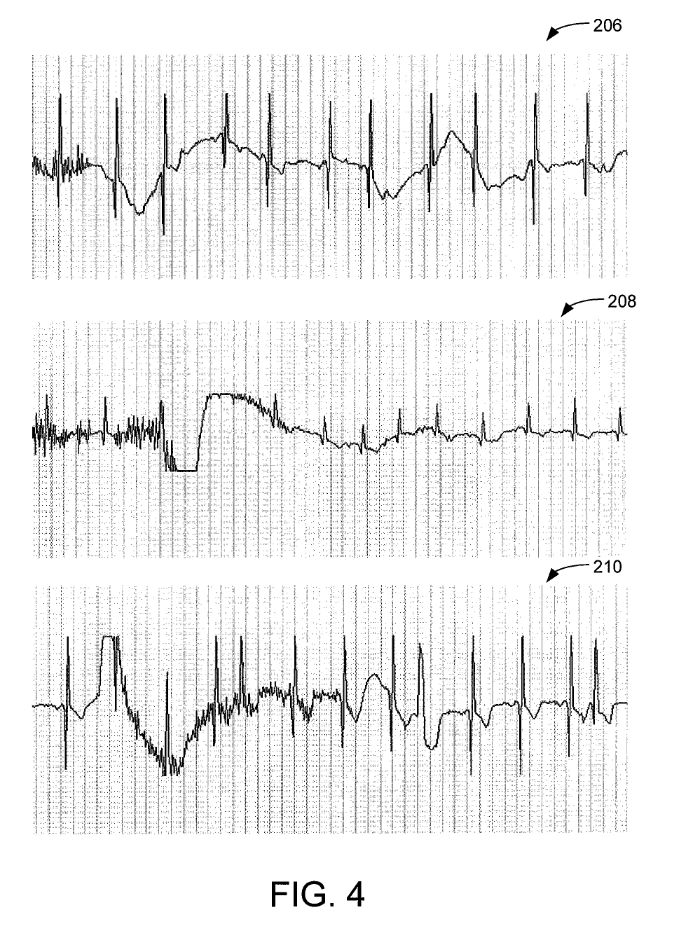

FIG. 4 illustrates exemplary subQ ECG signals in the presence of various electrode/tissue contact problems. More specifically, graph 206 illustrates contact problems resulting in noise and baseline wander, which might occur be due to patient movement. Graph 208 illustrates contact problems resulting in saturation, as well as noise and baseline wander, which again might occur due to patient movement. In this example, the ECG baseline changes to the extent the ECG signal is clipped (i.e. ECG signal saturation occurs.) Graph 210 provides yet another example of contact problems resulting in saturation, noise and baseline wander of the ECG signal.

Returning to FIG. 3, at step 212, the subQ device detects time-varying impedance signals (in response to the various impedance detection pulses delivered at step 202) and analyzes the impedance signals to detect: (a) acute instability of impedance indicative of intermittent electrode/tissue contact; (b) unacceptably high impedance (saturation) indicative of loss of electrode/tissue contact; (c) unacceptably low impedance (signal dropout) indicative of the presence of liquids surrounding at least one of the electrodes; and/or other poor electrode/tissue contact issues. Acute instability of impedance can be detected, for example, by tracking the impedance signal to detect frequent variations in the signal. The amount of variation in the signals can be compared against a predetermined threshold indicative of an unacceptably high amount of variation. Otherwise conventional techniques may be used for determining preferred or optimal values for such thresholds. Saturation of the impedance signal can be detected by identifying periods of time when the impedance signal reaches its largest detectable level (which typically depends upon the particular impedance detection circuitry used within the device.) Signal dropout of the impedance signal can be detected by identifying periods of time when the impedance signal drop to zero or to some other minimum value (which again typically depends upon the particular impedance detection circuitry used within the device.)

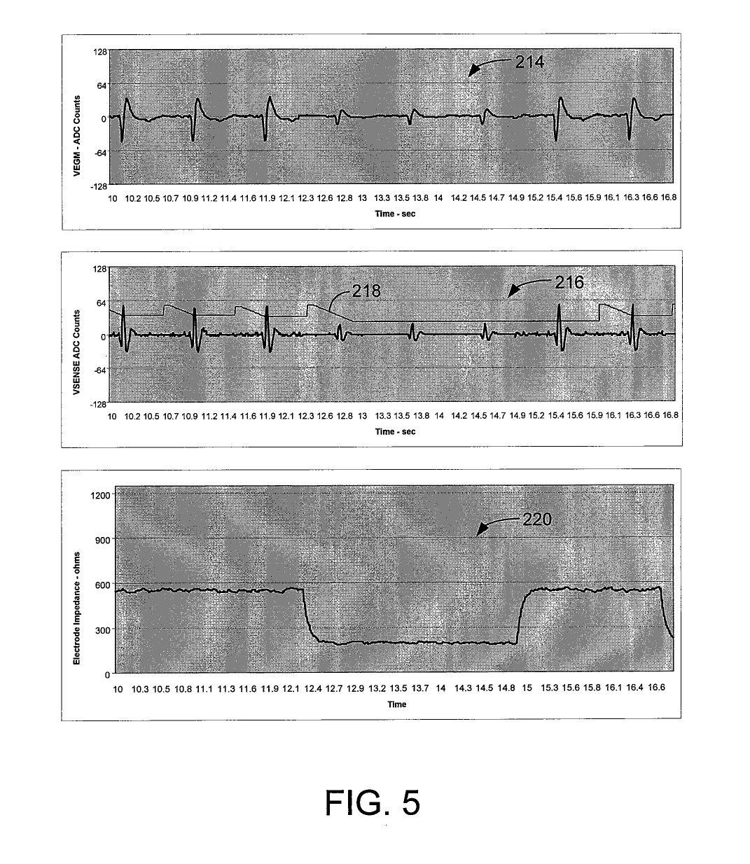

FIG. 5 illustrates subQ ECG signals that might be detected and stored by an ICM in the presence of various electrode/tissue contact problems, along with impedance (Z) variations indicative of impedance signal dropout. More specifically, graph 214 illustrates a subQ ECG (obtained on a VEGM channel, which is a wideband channel that the ICM uses to obtain the ECG to be stored) exhibiting a drop in R-wave magnitude likely due to the presence of liquids within the pocket of the device (where header and can electrodes are located.) Within graph 216 (obtained on a V-sense channel, which is a narrowband filtered channel that the ICM uses for detection), a trace 218 shows a time-varying R-wave detection threshold. Briefly, whenever an R-wave is detected, a refractory period is tracked and then the detection threshold increases to detect the next R-wave. The threshold decreases during individual cardiac cycles so as to permit detection of low amplitude R-waves. Once the threshold drops to a minimum level, no further reductions in the threshold are made. Hence, any R-waves having peak amplitudes below that minimal threshold are not detected by the device. As can be seen, in this particular example, the peak amplitude of the R-waves drops to the point that several of the R-waves are not detected in sequence, which could result in a false detection of asystole or bradycardia and the unnecessary storage of corresponding ECG data. Graph 220 illustrates concurrent impedance (Z) values, which show significant signal dropout indicating the likely presence of liquids surrounding at least one of the electrodes. (Liquids tend to conduct electrical signals much more readily than tissues and hence the amount of impedance drops.)

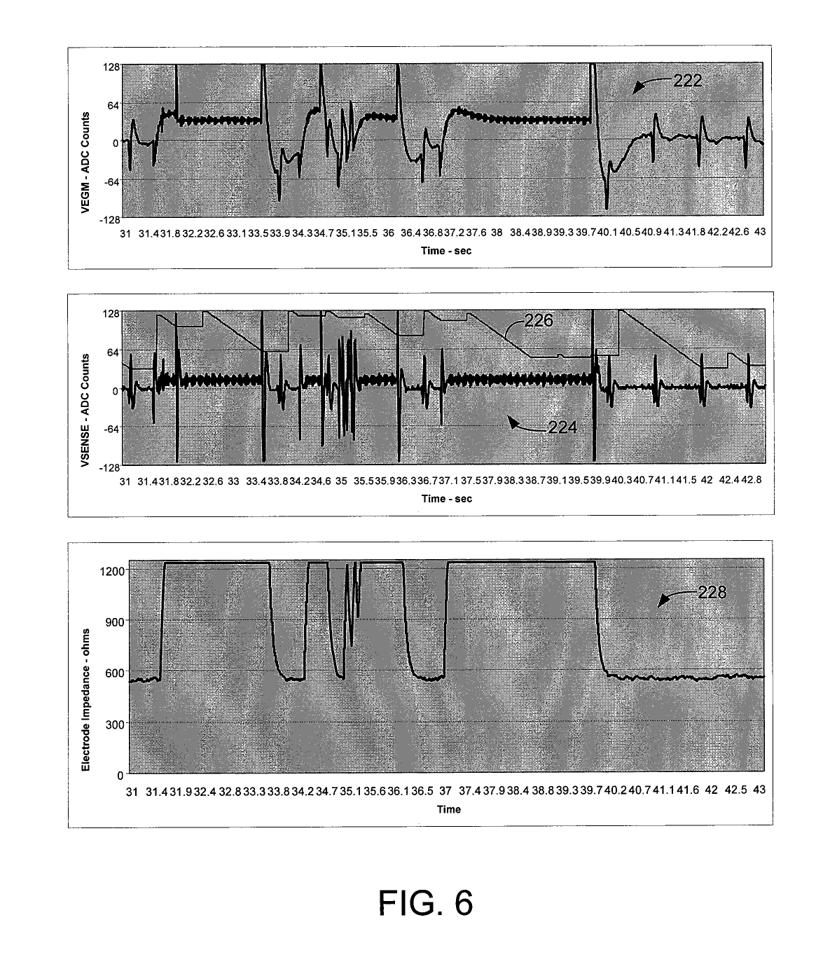

FIG. 6 illustrates another set of subQ ECG signals, along with impedance signals indicative of impedance signal saturation. More specifically, graph 222 illustrates a subQ ECG (again on a VEGM channel) exhibiting baseline wander. Within graph 224 (on a V-sense channel), a trace 226 again shows the time-varying R-wave detection threshold. As can be seen, in this particular example, the peak amplitude of the R-waves increases due to baseline wander to the point that the corresponding R-wave detection threshold is set too high. Several R-waves are again not detected, at various irregular intervals. The erratic detection of R-waves could result in false detection of AF and the unnecessary storage of corresponding ECG data. Graph 228 illustrates the concurrent impedance values, which show intermittent signal saturation indicating intermittent loss of electrode/tissue contact. (Air and/or vacuum does not readily conduct electrical signals and hence the amount of impedance increases sharply if there is a loss of electrode/tissue contact without intervening liquids.)

Returning again to FIG. 3, after the subQ device has detected and qualified any on-going electrode/tissue contact problems, the device at step 230 then inhibits the recording and/or transmission of ECG data and event markers during intervals of poor electrode/tissue contact, depending upon its programming. For example, the storage of ECG data can be inhibited in response to the detection of any of the problems identified in step 212 or, in some examples, in response to particular contact problems. If not currently inhibited, the device records ECG and event marker data at step 232 (and/or transmits the data to an external device in real-time) based on any arrhythmias detected at step 204. Still further, at step 234, the device records histograms of impedance data, analyze any trends therein, and assess changes in impedance due, e.g., to implant pocket maturation. (In this regard, proper tissue/electrode contact can be highly dependent on the mature state of the pocket and the tight fit of the device in the pocket. A loose device pocket might make the device especially prone to intermittent electrode/tissue contact.) Trend data can thereafter be downloaded using an external device for clinician review. In one particular example, a gradual increase in impedance up to a saturation level likely indicates a slow loss of electrode/tissue contact that might occur during pocket maturation if the electrodes are pulled away from surrounding tissues. In another example, a gradual decrease in impedance down to a signal dropout level likely indicates a slow accumulation of fluids around the electrodes. This type of information can be helpful to the clinician in assessing contact problems and in determining how best to address those issues. Still further, impedance trends can also be used at step 234 to detect circadian activities, such as bad electrode contact when the patient goes to sleep at night, turns over in bed, or runs on a treadmill while at the gym during the day, etc. The device can be programmed to inhibit recording or other functions during such intervals, at step 230.

Although not specifically shown in FIG. 3, following steps 232 or 234, processing continues at step 200 for sensing new subQ ECG signals within the patient. Also, note that the subQ device can also be equipped with additional noise-based or activity-based inhibitors that inhibit the recording of ECGs during periods of significant noise on the ECG signal (as might be caused by electromagnetic interference from power lines) or during periods when the patient is moving significantly (as might occur during patient exercise) as detected using an accelerometer. One advantage of the impedance-based monitoring techniques of the invention is that they can be used to qualify the nature of the poor electrode/tissue contact, as already explained. Also, electrode/tissue contact problems can occur during mundane tasks—such as when the patient turns over in bed or moves to a different sitting position—that are not necessarily accompanied by the sort of significant bodily motion or muscle activity which can be detected by conventional motion-detection accelerometers or which generate significant noise in the ECG signals.

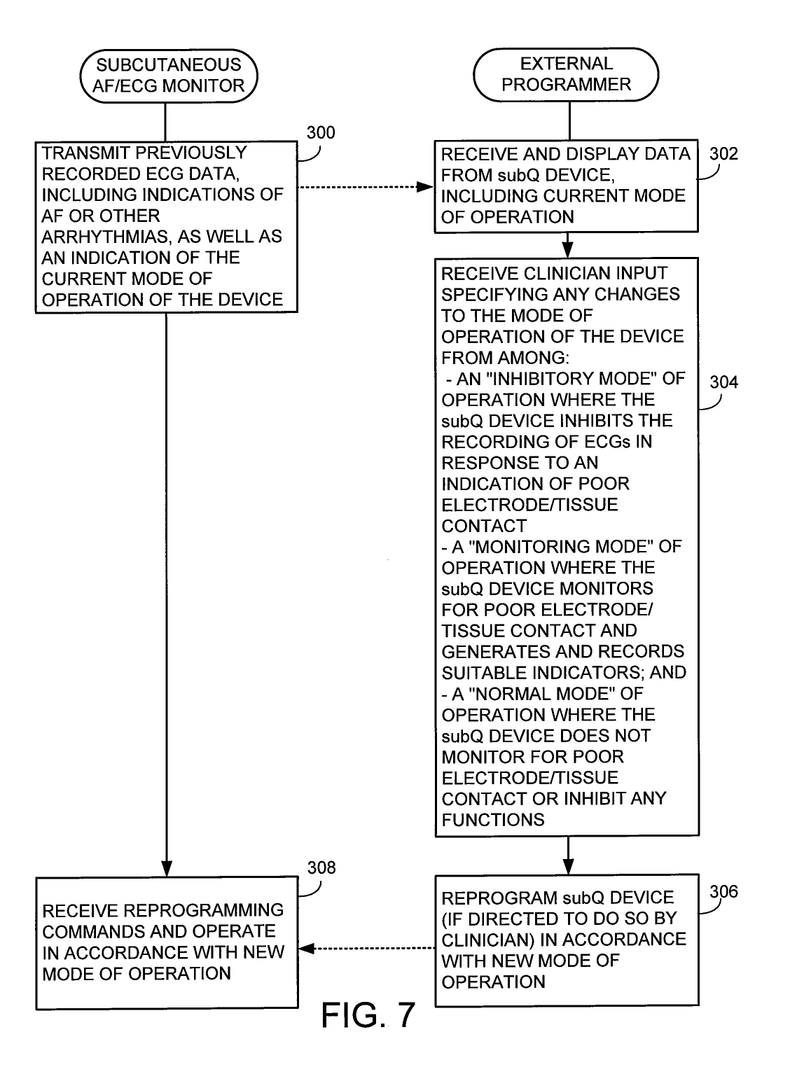

Turning now to FIG. 7, the operation of an external programming device in communication with the subQ device will be described. Beginning at step 300, the subQ device transmits previously recorded ECG data to the external programmer, including indications of AF, bradycardia or other arrhythmias that have been detected, as well as an indication of the current mode of operation of the device to the external programmer, such as whether the device is currently operating in an inhibitory mode. This information is received by the programmer at step 302 and displayed thereon for clinician review. At step 304, the programmer then receives clinician input specifying any changes to the mode of operation of the device from among: (a) the “INHIBITORY MODE” of operation where the subQ device inhibits the recording of ECGs in response to an indication of poor electrode/tissue contact; (b) the “MONITORING MODE” of operation where the subQ device monitors for poor electrode/tissue contact and generates and records suitable indicators but does not inhibit any functions; and (c) the “NORMAL MODE” of operation where the subQ device does not monitor for poor electrode/tissue contact nor inhibit any functions.

For example, if the device had been previously operating in the MONITORING MODE and detected several instances of poor electrode/tissue contact resulting in the recording of unneeded ECGs, the clinician may choose to switch the INHIBITORY MODE to prevent any further improper ECG recordings. The physician may also attempt to adjust the location of the subQ device and its electrodes within the patient to remedy the contact problem. As another example, if the device has been operating in the INHIBITORY or MONITORING MODE for some period of time but no instances of poor electrode/tissue contact have been detected, the clinician may choose to switch the device to the NORMAL MODE of operation. These are just some examples of possible modes of operation. Other modes might be programmable as well, such as modes that specify the particular device functions to be inhibited or which specify the particular contact issues to be detected.

At step 306, the programmer then sends suitable reprogramming commands to the subQ device to program the device to a new mode of operation (if directed to do so by the clinician.) The commands are received at step 308 by the subQ device, which then operates in accordance with the new mode of operation.

The above-described techniques can be implemented with a variety of subcutaneous implantable medical devices with extracardiac electrodes, in combination with a variety of external systems. In one particular implementation, the implantable medical device includes or comprises a Confirm™ monitor provided by St. Jude Medical, which is adapted for subcutaneous implant particularly within patients suspected of suffering episodes of AF. Also, in one implementation, the programmer includes or comprises a Merlin™ programmer also provided by St. Jude Medical.

### Exemplary Subcutaneous AF/ECG Monitor

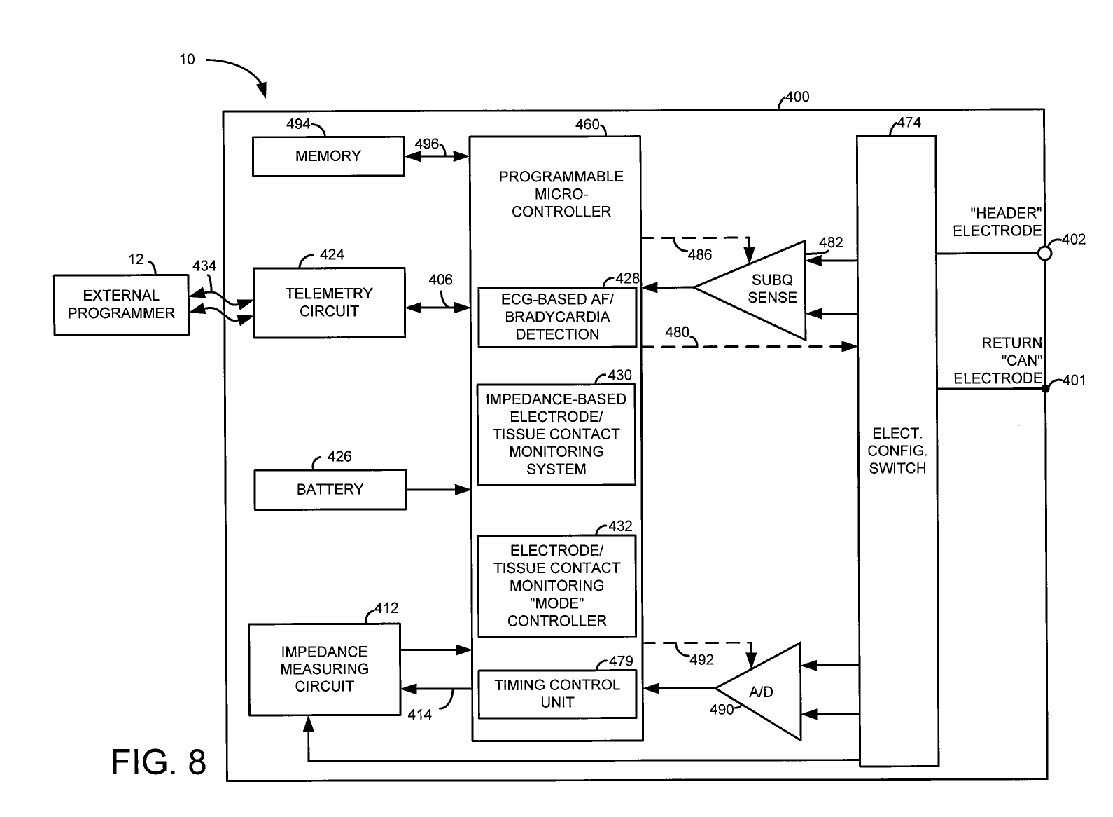

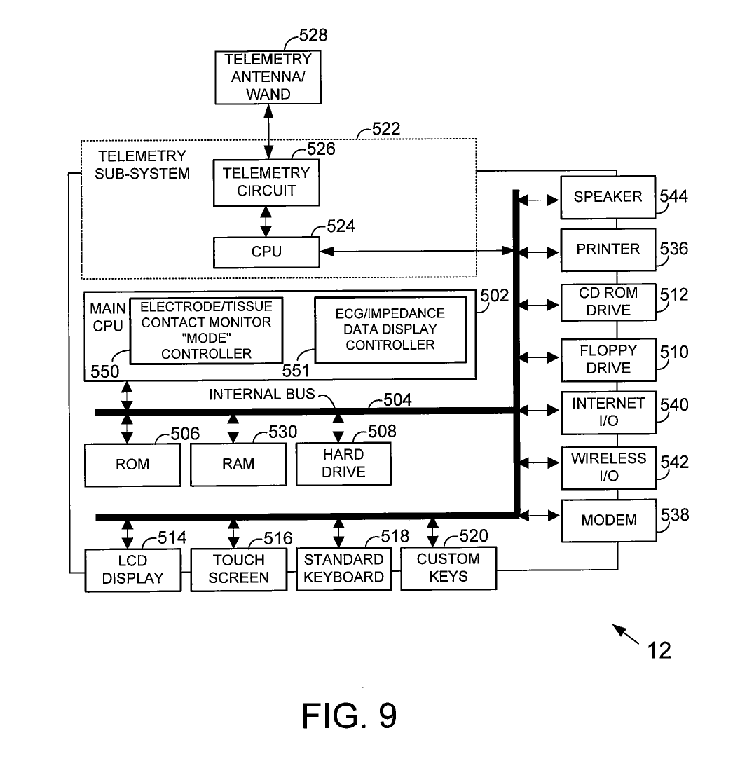

For the sake of completeness, internal components of an exemplary subQ ICM device 10 will now be summarized with reference to FIG. 8. This device is equipped to monitor for AF, as well as to detect electrode/tissue contact problems based on impedance. Housing 400 of monitor 10 includes a “can” return electrode 401 and an ECG sensing “header” electrode 402 mounted to (or connected to) the exterior housing of the device. Only two electrodes are shown, but additional electrodes or electrode terminals can be provided to accommodate additional sensing electrodes or sensing leads.

At the core of monitor 10 is a programmable microcontroller 460, which controls arrhythmia detection and electrode/tissue contact monitoring. The microcontroller 460 includes a microprocessor, or equivalent control circuitry, operative to detect arrhythmias such as AF and for monitoring for electrode/tissue contact problems and may further include random access memory (RAM) or read-only memory (ROM) memory, logic and timing circuitry, state machine circuitry, and I/O circuitry. Typically, the microcontroller 460 includes the ability to process or monitor input signals (data) as controlled by a program code stored in a designated block of memory. The details of the design and operation of the microcontroller 460 are not critical to the invention. Rather, any suitable microcontroller 460 may be used that carries out the functions described herein. The use of microprocessor-based control circuits for performing data analysis is well known in the art.

A switch bank 474 includes a plurality of switches for switchably connecting the ECG electrodes (assuming there is more than one) to the appropriate I/O circuits, thereby providing complete electrode programmability. A sense amplifier 482 is coupled to the ECG electrode(s) through switch bank 474 for detecting electrical cardiac activity. Sense amplifier 482 is capable of sensing signals in accordance with otherwise conventional techniques. The switch bank 474 determines the “sensing polarity” of the subQ cardiac signal by selectively closing the appropriate switches, as is also known in the art. In this way, the clinician may program the sensing polarity. Sense amplifier 482 preferably employs a low power, precision amplifier with programmable gain and/or automatic gain control and/or automatic sensitivity control, bandpass filtering, and a threshold detection circuit, known in the art, to selectively sense electrical signals of interest. The automatic gain control, if implemented, enables the device 10 to deal effectively with the difficult problem of sensing any low frequency, low amplitude signal characteristics. The gain control is actuated by the programmable microcontroller 460. The gains are controlled on sense amplifier 482 by the microcontroller using control line 486. The outputs of the sense amplifier are connected to microcontroller 460.

ECG signals and other sensed signals are also applied to the inputs of an analog to digital (A/D) data acquisition system 490. The gain of the A/D converter 490 is controlled by the microprocessor 460 by signals along control line 492 in order to match the signal amplitude and/or the resolution to a range appropriate for the function of the ND converter 490. The data acquisition system 490 is configured to acquire ECG signals, convert the raw analog data into a digital signal, and store the digital signals during episodes of arrhythmia for later processing and/or telemetric transmission to external device 12. The data acquisition system 490 is coupled to the ECG electrode 402 through switch bank 474 to sample cardiac signals. The microcontroller 460 is further coupled to a memory 494 by a suitable data/address bus 496, wherein the programmable operating parameters used by the microcontroller 460 are stored and modified, as required, in order to customize the operation of device 10 to suit the needs of a particular patient. Such operating parameters define, for example, the particular parameters to be used to detect AF or to control the mode of operation of the subQ device.

In this regard, the operating parameters of implantable device 10 may be non-invasively programmed into the memory 494 through telemetry circuit 424 in telemetric communication with external device 12 (which can be, e.g., a programmer, transtelephonic transceiver, bedside monitor, or a diagnostic system analyzer.) The telemetry circuit 424 is activated by the microcontroller 460 by a control signal 406. The telemetry circuit 424 advantageously allows subQ ECG electrograms and status information relating to the operation of device 10 (as contained in the microcontroller 460 or memory 494) to be sent to device 12 through an established communication link 434, and then on to a centralized processing system 14 (FIG. 1), where appropriate.

The implantable monitor additionally includes a battery 426 that provides operating power to all of the circuits shown in FIG. 8. The battery is capable of operating at low current drains for long periods of time for monitoring. The battery 426 also should have a predictable discharge characteristic so that elective replacement time can be detected.

For AF/bradycardia detection, the invention utilizes the sense amplifier to sense electrical signals to determine whether a cardiac rhythm is physiologic or pathologic. As used in this section, “sensing” is reserved for the noting of an electrical depolarization, and “detection” is the processing of sensed depolarization signals potentially in conjunction with other sensor input to establish a diagnosis of an arrhythmia. The timing intervals between sensed events (e.g., R-R intervals) are detected by a timing control unit 479 of microcontroller 460 and then classified by an ECG-based AF/bradycardia detection unit 428 by, for example, assessing R-R interval stability (for detecting AF) or by assessing the ventricular rate (for detecting bradycardia.) The timing control unit can also track the aforementioned refractory periods.

The microcontroller also includes an impedance-based electrode/tissue contact monitoring system 430, which is operative to detect an indication of poor electrode/tissue contact based on impedance signals sensed using an impedance measuring circuit 412 in accordance with techniques described above. An electrode/tissue contact monitoring “mode” controller 432 is provided to control the mode of operation of the electrode/tissue contact monitoring system, in accordance with the aforementioned modes of operation that are programmed into the device via external programmer 12.

The microcontroller is thereby operative to control at least one device function in response an indication of poor electrode/tissue contact, such as by inhibiting the recording of ECG data in memory 494, or by controlling the generation and transmission of diagnostic data and warnings to the external programmer, as already explained.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, application specific integrated circuits (ASICs) or the like.

### Exemplary External Programmer Device

FIG. 9 illustrates pertinent components of an external programmer 12 for use in displaying information received from the subQ device of FIG. 8 and for performing the above-described programming techniques. Generally, the external programmer permits a user (such as a clinician) to retrieve and display information received from the implantable monitor such as AF detection event markers, ECG data and device diagnostic data. The external programmer also displays various reprogramming commands for allowing the clinician to easily reprogram the operation of the subQ device.

Now, considering the components of external programmer 12, operations of the programmer are controlled by a CPU 502, which may be a generally programmable microprocessor or microcontroller or may be a dedicated processing device such as an ASIC or the like. Software instructions to be performed by the CPU are accessed via an internal bus 504 from a ROM 506 and RAM 530. Additional software may be accessed from a hard drive 508, floppy drive 526, and CD ROM drive 512, or other suitable permanent mass storage device.

In use, a telemetry antenna or wand 528 operates to receive any signals sent from the subQ device, such as the aforementioned electrode/tissue contact problem warnings. Antenna 528 is connected to a telemetry subsystem 522, which includes its own CPU 524 for coordinating the operations of the telemetry subsystem. Main CPU 502 of programmer communicates with telemetry subsystem CPU 524 via internal bus 504. Telemetry subsystem includes a telemetry circuit 526 connected to the telemetry antenna, which, receives and transmits signals electromagnetically from a telemetry unit within the subQ device. If a telemetry wand is used, the wand is placed over the chest of the patient near the subQ device to permit reliable transmission of data between the telemetry wand and the device.

Upon receipt of an indication of an electrode/tissue contact problem from the subQ device, main CPU 502 displays warning information to the user (e.g. clinician) via an LCD display 514 or other suitable computer display device or other output device (such as a speaker 544) to alert the clinician to a possible sensing problem within the patient. The CPU then displays ECG data, event markers, impedance data, etc., under the control of ECG/impedance data display controller 551. The clinician can then reprogram the subQ device, if warranted. The reprogramming commands are generated by an electrode/tissue contact monitor “mode” controller 550 then transmitted to the device via the telemetry wand.

Additional programmer components that can be exploited include a speaker 544, an Internet input/output (I/O) unit 540, a wireless I/O unit 542, a modem 538 or other suitable I/O system to permit direct transmission of data to other external devices via the public switched telephone network (PSTN) or other interconnection lines, such as a T1 lines or fiber optic cables. Suitable information may be sent, e.g., to one of the aforementioned Merlin systems.

Note that the external programmer may also receive other types of diagnostic information from the subQ device via the telemetry system for display, such device diagnostic data, which can include, e.g., information representative of the operation of the implanted device such as battery voltages, battery recommended replacement time (RRT) information and the like. Data retrieved from the subQ device is stored either within a RAM 530, hard drive 508 or within a floppy diskette placed within floppy drive 526. Additionally, or in the alternative, data may be permanently or semi-permanently stored within a digital media disk.

As with the microcontroller of the subQ device, the various components of the microcontroller of the external device may be implemented as separate software modules or the modules may be combined to permit a single module to perform multiple functions. In addition, although shown as being components of the microcontroller, some or all of these components may be implemented separately from the microcontroller, ASICs or the like.

Still further, the principles of the invention may be exploiting using other implantable systems or in accordance with other techniques. Thus, while the invention has been described with reference to particular exemplary embodiments, modifications can be made thereto without departing from scope of the invention. Note also that the term “including” as used herein is intended to be inclusive, i.e. “including but not limited to.”

What is claimed is:

1. A method for use with an implantable subcutaneous medical device for subcutaneous implant within a patient wherein the device employs extracardiac sensing electrodes for contact with patient tissue, the method comprising:
detecting subcutaneous impedance signals using the extracardiac sensing electrodes of the subcutaneous device;
detecting an indication of poor electrode/tissue contact within the subcutaneous impedance signals; and
controlling at least one device function in response the indication of poor electrode/tissue contact.

2. The method of claim 1 wherein detecting impedance signals using the sensing electrodes of the subcutaneous device includes:
tracking a cardiac refractory period;
delivering impedance detection pulses during the refractory period; and
detecting subcutaneous impedance during the refractory period.

3. The method of claim 1 wherein detecting impedance signals using the sensing electrodes of the subcutaneous device includes:
delivering a stream of impedance detection pulses at a sufficiently low amplitude regardless of refractory periods to avoid cross talk on ECG; and
detecting impedance based on the stream of impedance detection pulses.

4. The method of claim 1 wherein detecting an indication of poor electrode/tissue contact is performed to detect contact problems resulting in one or more of noise, baseline wander, saturation, and signal dropout within an electrocardiac signals sensed by the device.

5. The method of claim 1 wherein detecting an indication of poor electrode/tissue contact is performed to detect an acute instability of impedance indicative of intermittent electrode/tissue contact.

6. The method of claim 1 wherein detecting an indication of poor electrode/tissue contact is performed to detect unacceptably high impedance indicative of loss of electrode/tissue contact.

7. The method of claim 1 wherein detecting an indication of poor electrode/tissue contact is performed to detect unacceptably low impedance indicative of the presence of liquids surrounding at least one of the electrodes.

8. The method of claim 1 wherein controlling at least one device function in response the indication of poor electrode/tissue contact includes generating an indicator for indicating poor electrode/tissue contact.

9. The method of claim 8 wherein the indicator is an event marker for storage in memory.

10. The method of claim 8 wherein the indicator is a signal transmitted substantially in real-time from the device to an external system.

11. The method of claim 8 wherein indicator identifies the particular type of poor electrode/tissue contact.

12. The method of claim 1 wherein the device is equipped to store cardiac signal data sensed using the electrodes and the step of controlling at least one device function in response the indication of poor electrode/tissue contact includes inhibiting the storage of cardiac signal data during an interval of poor electrode/tissue contact.

13. The method of claim 1 wherein the device is equipped to identify an arrhythmia based on an analysis cardiac signal data sensed using the electrodes and the step of controlling at least one device function in response the indication of poor electrode/tissue contact includes inhibiting the identification of arrhythmias during an interval of poor electrode/tissue contact.

14. The method of claim 1 further including the step of storing a histogram of impedance signals.

15. The method of claim 1 further including the step of trending the impedance signals over time to assess changes in impedance due to one or more of: implant pocket maturation and circadian activities.

16. A system for use with an implantable subcutaneous medical device for subcutaneous implant within a patient wherein the device employs extracardiac sensing electrodes for contact with patient tissue, the system comprising:
a subcutaneous impedance detection system operative to detect impedance signals using the extracardiac electrodes of the subcutaneous device; and
an electrode/tissue contact monitoring system operative to detect an indication of poor electrode/tissue contact based on the subcutaneous impedance signals; and
a controller operative to control at least one device function in response an indication of poor electrode/tissue contact.

17. The system of claim 16 wherein the device is used in conjunction with an external monitoring device and wherein the external monitoring device is operative to control the implantable subcutaneous medical device to operate in an inhibitory mode of operation wherein the implantable subcutaneous medical device inhibits the recording of cardiac signals in response to an indication of poor electrode/tissue contact.

18. The system of claim 16 wherein the device is used in conjunction with an external monitoring device and wherein the external monitoring device is operative to control the implantable subcutaneous medical device to operate in a monitoring mode of operation wherein the implantable device monitors for poor electrode/tissue contact and generates indicators.

19. The system of claim 16 wherein the device is used in conjunction with an external monitoring device and wherein the external monitoring device is operative to control the implantable subcutaneous medical device to operate in an normal mode of operation wherein the implantable device does not monitor for poor electrode/tissue contact.

20. A system for use with an implantable subcutaneous medical device for subcutaneous implant within a patient wherein the device employs extracardiac sensing electrodes for contact with patient tissue, the system comprising:
means for detecting subcutaneous impedance signals using the extracardiac sensing electrodes of the subcutaneous device;
means for detecting an indication of poor electrode/tissue contact within the subcutaneous impedance signals; and
means for controlling at least one device function in response the indication of poor electrode/tissue contact.
